(19) United States
(12) Patent Application Publication
(10) Pub. No.: US 2009/0018517 A1
(43) Pub. Date: Jan. 15, 2009

(54) COMPOSITE ABSORBENT MATERIAL WITH WATER-SOLUBLE BONDING AGENTS, PRODUCTS MADE OF SAID MATERIAL NAD METHOD FOR ITS MANUFACTURE

(76) Inventors: Riccardo Cecconi, Prato (IT); Barbara Bulleri, Pistoia (IT); Chiara Allegrini, Lucca (IT)

Correspondence Address: McGLEW AND TUTTLE, P.C., Scarborough Station, Scarborough, NY, 10510-9227, US

(21) Appl. No.: 11/776,191

(22) Filed: Jul. 11, 2007

(51) Int. Cl. A61F 13/539; A61F 13/53; B32B 37/02

(52) U.S. Cl. 604/365; 604/367; 156/276

(57) ABSTRACT

A method is described for the manufacture of a composite absorbent material comprising the following stages: superabsorbent particles (P) are distributed on at least one first textile web (V1); said at least one textile web and said particles are impregnated with a highly water-soluble bonding agent; said bonding agent is dried and the particles are induced to adhere to said at least one textile web.

### TECHNICAL FIELD

This invention relates to a method for manufacturing an absorbent textile web-like material, i.e. in sheet form, containing particles or powders of at least one superabsorbent material. The invention also relates to a material made according, to said method, e.g. a sanitary towel or diaper, containing said product.

### STATE OF THE ART

In the manufacture of absorbent materials, such as diapers for infants, women's sanitary towels, incontinence pads, and so on, it is essential to achieve structures with a great capacity for absorption. A further requirement of the technology for manufacturing such products concerns the thickness of the end product. This must be as thin as possible to reduce the discomfort to the person wearing it, without its small dimensions and limited thickness having a negative effect on its capacity for absorption.

For this purpose, studies have focused on materials called superabsorbent polymers (SAP), or other highly-absorbent particles, e.g. sodium polyacrylates, polysaccharides, and so on. These particles at least partially replace the bulky layers of wadding, or integrate the latter's function, thereby assuring the end product a greater capacity for absorption.

In the present description, reference is frequently made to superabsorbent polymer particles, or SAP particles, but it is important to be aware that the invention is not restricted to these products alone, but also covers the use of any type of material in the form of a powder, fibers, granules, or other particles in general, that have comparable high-absorbance features.

The SAP particles, in powder, fiber or other form, must be evenly distributed and anchored within the fibrous structure of the product. Their anchorage is a crucial aspect, because the superabsorbent particles must not escape during the manufacturing process or when the product is in use. Moreover, they must not be allowed to collect randomly in certain parts of the end product, since this would detrimental to its absorbent properties. A build-up of superabsorbent particles gives rise to a defect in the end product, because certain areas are scarcely absorbent, while there are clumps of superabsorbent particles elsewhere.

Various techniques have been suggested for distributing the superabsorbent particles and anchoring them to the structure of the absorbent product.

WO-A-03/065951 describes methods and devices for applying and anchoring SAP particles in a carded textile structure. In some of the embodiments described in this patent, a magnetic field is used to make the SAP particles penetrate the structure of the textile web, which may be consolidated—i.e. converted into a nonwoven fabric—beforehand by means of a hydro-entanglement process or other suitable technology.

WO-A-03/073971 describes methods for manufacturing sheet products with superabsorbent polymer particles inserted in a sandwich comprising several textile layers.

U.S. Pat. No. 5,585,170 describes a method that uses an electric field to distribute SAP powders and apply it to a substrate passing through the electrostatic field.

EP-A-1.154.061 describes a superabsorbent composite material and a method for its manufacture. In this procedure, a web of unbonded fibers is prepared and then spread with a bonding component mixed with super-absorbent particles. The product is then dried.

EP-A-333228 and U.S. Pat. No. 4,655,757 describe methods for manufacturing a product with a so-called melt-blown process, starting from extruded continuous filaments. In some of the examples provided, a superabsorbent polymer powder is included in the fibrous structure. The superabsorbent polymer powder is mixed together with cellulose fibers, which are then distributed over a preformed web of fibers created using the melt-blowing technique.

EP-A-719531 describes a composite fibrous product containing SAP particles. These particles are anchored to hydrophilic fibers that constitute the absorbent material.

In “Superabsorbent Composite Acquisition Layers”, NONWOVENS WORLD, February-March 2003, page 47 onwards, by Evan Koslow, an absorbent composite product is described, wherein a structure of superabsorbent particles is created between two layers (at least one of which is a porous nonwoven), said particles adhering to each other due to the effect of a bonding agent composed of a thermoplastic resin powder. The manufacturing process involves mixing the SAP particles with the thermoplastic powder and distributing the mixture over a bottom layer. Then the second layer is placed on top to form a sort of “sandwich”, with the superabsorbent particles inserted between the two, top and bottom layers of composite structure. The “sandwich” is heated to melt the thermoplastic powder and thus anchor the superabsorbent polymer particles to each other and to the top and bottom layers of the structure.

This procedure gives rise to a thin product capable of rapidly absorbing large quantities of fluid and thus particularly suitable for manufacturing products for personal hygiene, such as women's sanitary towels. It has some drawbacks, however. Firstly, the anchorage of the SAP particles is not ideal, especially near the edges of the composite product. Moreover, the procedure is complicated by the need to mix the superabsorbent particles with the thermoplastic resin powder before their application.

EP-A-463716 describes a method for manufacturing an absorbent structure, in which a layer of superabsorbent powders is inserted between two fibrous layers. A bonding agent is sprayed onto one or both sides of the resulting structure. The final structure is not well united, however, because the bonding agent does not reach the inside and does not bond the two fibrous layers together. The product tends to delaminate, i.e. the two fibrous layers tend to separate. Moreover, the lack of any anchorage between the superabsorbent particles and the fibers gives rise to a loss of powders, particularly along the edges and along the lines where the product is cut.

GB-A-2004201 describes a method for manufacturing an absorbent structure wherein a layer of superabsorbent powders is distributed over a fibrous web. A bonding agent is then sprayed over the powders to make it adhere to the fibrous layer.

### OBJECTS AND SUMMARY OF THE INVENTION

The object of the present invention is to provide a method enabling the manufacture of an absorbent composite containing superabsorbent particles within a textile structure, that fulfils the requirements of a great capacity for absorption, limited thickness, low production costs and efficient retention of the superabsorbent particles within the textile structure.

These and other objects and advantages, that will become clear to those skilled in the art from reading the following description, are substantially achieved using a procedure comprising the following steps:
distributing particles of a superabsorbent material over at least one textile web;
impregnating the full thickness of the particles and textile web with a bonding agent highly soluble or dispersible in water;
adhering the particles to said at least one textile web by means of said bonding agent.

The highly water-soluble or dispersible bonding agent is preferably used alone and may be composed of a mixture of several components, all highly soluble or dispersible in water. However, according to a possible embodiment of the invention, a first bonding agent (in the sense of a single component or mixture of components that dissolve or disperse rapidly in water) may be combined with a second bonding agent (comprising a single component or a mixture of components) of limited solubility or dispersibility in water. This second bonding agent may be included in proportions varying between 0% and 50%, and preferably from 10% to 30% of the total weight of the bonding agent contained in the end product. When used, its purpose is to keep the fibers and superabsorbent particles together for some time, even after they have come into contact with the body fluid. This may be important in some cases, to retain a greater cohesion in the end product, e.g. in a sanitary towel.

The bonding agent may be water-based, in which case it can be dried and subsequently crosslinked by heating, though other bonding components can be used that may not need hot crosslinking. The bonding agent may be a resin emulsion, adhesive, or other material. To penetrate the full thickness of the textile web, a relatively large amount of bonding agent is needed. This achieves a very effective stabilization of the product, and possibly also a cohesion of the fibers forming the web, which may be an unbonded web, such as (and advantageously) an unbonded carded web. The term bonded is used to mean a web of fibers that has undergone a process in order to consolidate said fibers together, whereas the term unbonded describes a web that has not been submitted to any specific process, e.g. a chemical, thermal or mechanical process, to consolidate its fibers together.

Using the bonding agent to impregnate the unbonded (or, in other words, unconsolidated) fibrous layer enables both the cohesion of the web and the effective anchorage of the powders to be achieved in a single step. The capacity of the bonding agent to disperse or dissolve in water prevents the large quantity of bonding agent employed from having any negative impact on the absorbent properties of the end product.

In fact, when the product is inserted, for instance, in a sanitary towel or diaper, the body fluid that it is required to absorb rapidly causes a considerable proportion of the bonding agent to disperse or dissolve, thus releasing the superabsorbent particles, which are consequently free to absorb large amounts of fluid.

Suitable water-soluble bonding agents or adhesives may be of natural (starches) or synthetic origin. Among these, the polyvinyl alcohol resins are particularly worth considering, albeit without overlooking other suitable water-soluble resins. Among others, the following water-soluble adhesives or water-soluble hot-melt adhesives available on the market are worth mentioning:
1) Trade name: Nearvil LC 50 Composition: polyvinyl alcohol polymer Manufacturer: Nearchimica SPA (Italy)
2) Trade name: HYDROPELLET LTF Composition: polyvinyl alcohol based polymer Manufacturer: Idroplax S.r.l. (Italy)
3) Trade name: Carbosol 25 Composition: carboxymethylcellulose Manufacturer: Lamberti S.p.A. (Italy)
4) Trade name: CYCLOFLEX 34-625A Composition: polyvinyl alcohol based polymer Manufacturer: National Starch & Chemical (USA)
5) Trade name: XTH 81820-1 Composition: polyvinyl alcohol based polymer Manufacturer: Bostik-Findley Nederland BV (Netherlands)
6) Trade name: PVA 15/79 Composition: polyvinyl acetate with 79% OH group substitution Manufacturer: Lamberti S.p.A. (Italy)
7) Trade name: Lamcol WN 200 Composition: polyvinyl acetate with medium grade (30%) OH group substitution Manufacturer: Lamberti S.p.A. (Italy)

These bonding agents or adhesives can be dissolved in water and made foamable to make it easier to spread them with padder systems, but soaking, spraying or hot-melt spreading methods, or a printing roll or other such method can also be used, providing the bonding agent or adhesive penetrates the full thickness of the product so as to ensure a reliable anchorage of the superabsorbent particles as well as the cohesion of the fibers (in the case of unbonded webs being used) and the cohesion of the various webs comprising the material, if this includes a composite structure comprising several layers.

Suitable bonding agents or adhesives shall be quick to dissolve or disperse in water. This ensures that the product, inserted in an absorbent article such as a sanitary towel or diaper, can rapidly acquire and absorb large quantities of body fluid despite the presence of the bonding agent, which dissolves or disperses quickly enough to enable the fluid (blood or urine) to penetrate through the fibers and reach the superabsorbent polymer particles. The bonding agent is rapidly removed from the surface of these particles, which can thus absorb the fluid and expand freely.

The term highly-soluble bonding agent is used generally to mean a bonding agent that dissolves in water at such a rate as to permit the rapid absorption of body fluids by an absorbent product made with said bonding agent. According to a particularly advantageous embodiment of the invention, the bonding agent shall dissolve at ambient temperature at such a rate that at least 40% of the bonding agent covering the grains or particles of superabsorbent material dissolves within ten seconds or less, preferably within four seconds or less, in a simulation based on acquisition and rewet tests (described below).

In a particularly advantageous embodiment of the invention, the superabsorbent material is polysaccharide-based, because this material is biodegradable. A material of this type might be, for instance, LYSORB, manufactured by Lysac Technologies Inc. (Canada).

Alternatively—or in combination with the polysaccharides—other superabsorbent materials, such as polyacrylates, may also be used. Among these, the following products were found suitable for use in the present invention:
1) Trade name: FAVOR SXM 9140 Composition: surface crosslinked sodium polyacrylate Manufacturer: Degussa, which is part of Stockhausen GmbH & Co. KG, (Germany)
2) Trade name: AQUAKEEP HP 200 Composition: surface crosslinked sodium polyacrylate Manufacturer: ATOFINA Italia S.r.l.
3) Trade name: EK 1055 Composition: sodium polyacrylate with gradient crosslinking Manufacturer: DOW Deutschland GmbH & Co KG (Germany)

A further category of potential superabsorbent materials is represented by hydroxyethylcellulose- and/or carboxymethylcellulose-based products. For example, a hydroxyethylcellulose- and carboxymethylcellulose-based product crosslinked with divinyl sulfone proved suitable for application in the present invention.

Using the bonding agent to bond the fibers or filaments forming the textile structure makes it unnecessary to mix the SAP particles and thermoplastic powders before their application, thereby simplifying the production plant and manufacturing process. In addition, for the application and anchorage of the superabsorbent particles, the method according to the invention thus uses a production stage that may (in some cases, at least) already be planned for other purposes, i.e. to bond the fibers forming the textile web or layer and/or to join several textile webs or layers together.

In a preferred embodiment of the invention, the superabsorbent particles are distributed between two textile webs or layers, forming a sort of “sandwich”. Each layer can be composed of several webs. In this preferred embodiment of the invention, the method comprises the following steps:
providing a first web of textile fibers;
distributing a certain amount of superabsorbent particles on one side of said first web of textile fibers;
applying a second web of textile fibers over the superabsorbent particles;
impregnating the structure comprising the first and second layers of textile web and the superabsorbent particles contained between said two webs with the bonding agent;
cross-linking the bonding agent.

It is important to understand that the two textile fiber webs can also be composed of two portions of the same product, folded one over the other. Moreover, each web may itself be composed of several components or layers.

Impregnation is suitably and preferably achieved by immersion, using a two- or three-cylinder padder machine, for instance, so that the bonding agent penetrates the full thickness of the product, impregnating the fibers and the particles of superabsorbent material.

The impregnation of the two textile layers and of the particles placed in between them guarantees the mutual anchorage of the two layers and the anchorage and stabilization of the SAP particles. If the webs are composed of unbonded fibers, said impregnation also ensures the bonding of the fibers. An excellent anchorage of the powders or particles of superabsorbent material is assured even along the edges of the end product.

By comparison with known methods, moreover, impregnation guarantees a more reliable adhesion of the single SAP particles to the fibers forming the textile layers or webs. The anchorage of the single particles is important because their dimensions are often of the same order of magnitude as the thickness of the textile webs or layers. Typically, the webs are around 600 micrometers thick, for instance, while the particles of superabsorbent material have diameters ranging, for example, from 50 to 850 micrometers, because they are ungraded powders. Any excessively large or excessively small particles that are not adequately anchored to the textile structure escape from the sandwich created by the two webs. The anchorage achieved by impregnation prevents this defect.

According to an advantageous embodiment, the bonding agent can be composed of a latex, e.g. a resin in an emulsion with water, or air and water, i.e. in a foam.

Although the use of non-aqueous solvents or emulsions is not ruled out, using aqueous emulsions makes the procedure more straightforward and more environment-friendly, since it avoids the need to recover the solvents. Generally speaking, however, the bonding agent may also be composed of a material other than a crosslinkable resin emulsion. It may generally be any product that serves the purpose of anchoring the superabsorbent particles and that exists in a physical state (liquid or foam) suitable for impregnating the web-like material.

It has been demonstrated that impregnation with a resin emulsified in water does no damage to the superabsorbent particles. In fact, although these particles characteristically swell as they absorb water very rapidly and in large quantities, this swelling of the SAP particles is reversible, particularly when care is taken to complete the resin-coating/drying-crosslinking process fairly quickly in order to avoid the development of macrogels, i.e. clumps of particles. As a consequence, as it dries, the material returns to its original conditions, i.e. with the SAP particles containing no water and ready to absorb fluids again.

In an advantageous embodiment of the method according to the invention, the one or more textile webs or layers and the superabsorbent polymer particles are dried in an oven to eliminate their water content and complete the crosslinking of the resin. As mentioned earlier, this may also be a self-crosslinking resin, in which case heating is required for the sole purpose of eliminating the water and completing the crosslinking process.

The textile webs or layers can be composed of staple fibers, or they may also be made of continuous filaments, e.g. using the spun-bonded or melt-blown technology.

According to a preferred embodiment, the webs are unbonded carded webs, which are bonded by impregnation with the bonding agent, thereby achieving a particularly efficient production process.

The advantages gained from the method according to the invention, especially when the one or more fiber webs are carded webs, consist in a greater softness and a considerably lower weight by comparison with the conventional structures. Both these features are particularly useful when the product is used as a semi finished component in the manufacture of absorbent articles, such as diapers and women's sanitary towels.

Conventional absorbent sheets, typically obtained using air-laid techniques, have to assure a weight ratio between the fiber (cellulose pulp) and the superabsorbent powder of at least 2:1, which makes it necessary to produce a material with a greater concentration of cellulose pulp than might otherwise be necessary. In fact, the fibers comprising the air-laid web are very short and have a limited capacity for retaining the superabsorbent powder.

Conversely, the method according to the invention enables the weight ratio between the fiber and superabsorbent particles to be reduced to 1:4, or rather to 1:3.5, or preferably to approximately 1:3, thanks to the greater efficiency of the system for anchoring the superabsorbent particles to the fibrous structure. Thus, the weight ratio between the fibers and the superabsorbent particles can vary between 1:0.8 and 1:4, and preferably between 1:1 and 1:3.5 or, even more preferably, between 1:1.5 and 1:3.5, with optimal values coming between 1:2 and 1:3.2.

Typically, when webs made of carded fibers are used, suitable webs have a weight coming between 5 and 150 g/m², and preferably between 5 and 120 g/m² or, more preferably, between 8 and 100 g/m², and especially between 8 and 80 g/m², or between 8 and 60 g/m². Using two carded webs coupled together, the total weight of the product advantageously arrives at between 10 and 160 g/m², and preferably between 16 and 120 g/m²; all said weights are expressed without considering the weight of the bonding agent and superabsorbent particles. A third web of nonwoven fabric or tissue paper, typically weighing between 5 and 50 g/m², and preferably between 15 and 25 g/m², may complete the textile structure of the product. It is preferable for the global weight of the textile structure (without considering the weight of the superabsorbent material) to be less than 150 g/m² or more preferably, less than 120 g/m².

A structure obtained in this way can incorporate up to approximately 300 g/m², and typically from 2 to 300 g/m² of superabsorbent powders or particles, and preferably from 10 to 200 g/m² of superabsorbent powders or particles or, more preferably, from 10 to 100 g/m² of superabsorbent powders or particles. Particularly advantageous weights lie between 50 and 100 g/m². Vice versa, the typical weights of absorbent structures obtained according to currently-used techniques, which use webs or layers of cellulose fibers made using air-laid technology, will be around 170 g/m² for the fibers and 60 g/m² for the superabsorbent material.

The textile structure with the superabsorbent particles or powders distributed between the two webs is impregnated with quantities of bonding agent or adhesive that can vary, for instance, between 5% and 50% of the weight of the end product, and preferably between 10% and 50% or, more preferably, between 15% and 35% or, even more preferably, between 15% and 25% of the weight of the end product.

In a particularly advantageous embodiment of the invention, the superabsorbent particles are placed between two webs of unbonded fibers, with the lower web resting on a sheet of nonwoven fabric, the main purpose of which is to prevent the SAP particles from escaping due to gravity through the unbonded fibrous structure of the web on which they are distributed.

In addition to the superabsorbent particles, other products, e.g. deodorant powders or other components, may be added using the same method.

According to a particularly advantageous embodiment of the invention, provision can be made to include an auto-expanding resin in the material. This may be added, for instance, in proportions varying between 2 and 95%, and preferably between 2 and 50% of the weight of the bonding agent. Among the types of expanding resin that it is possible to use, Expancell 051 WU and Expancell 091 WU manufactured by Schonox GmbH (Germany) can be mentioned.

These resins are typically polymer resins in spherical form, containing isobutane or another equivalent gas. In contact with a source of heat at a certain temperature (which depends on the type of polymer resin used), the gas expands and the polymer swells to up, to four times its original volume. So the auto-expanding resin swells during the hot drying and/or crosslinking of the bonding agent with which the structure is impregnated. This gives rise to an increase in the volume of the structure as a whole and offers an advantage in terms of fluid acquisition rate when the product is used in a sanitary towel or other such product, since the open textile structure reduces the time it takes to absorb the fluids.

The expanding resin particles or microspheres can be mixed with the fibers or superabsorbent particles or, preferably, with the bonding agent. When the particles of auto-expanding resin are mixed with the bonding agent, they come to be distributed in a substantially uniform manner throughout the textile structure of the product, so that their expanding effect on the fibers effectively involves the entire structure.

The choice of expanding resin depends on the temperature to which the product is submitted, for instance, to dry it and/or induce or complete the crosslinking of the bonding agent. Resins can be used either that expand at the maximum temperature applied, or that first expand and then collapse due to an excessive dilation of the microparticles or microspheres as a consequence of the high temperature applied. The cavities created inside the fibrous structure thus remain substantially empty because the polymer film around the gas in the auto-expanding particle collapses and shrinks but the surrounding fibers have in the meantime become bonded due to the crosslinking of the bonding agent.

Although the basic structure of the absorbent material according to the invention is composed of at least one textile web (or preferably two textile webs coupled together) and of the particles adhering thereto (or sandwiched between the two webs), this does not rule out the possibility of the material being of a more complex, composite structure. To be more precise, it may include more than two layers or webs. To give it greater mechanical strength during its manufacture, for instance, and/or to avoid the superabsorbent particles escaping when they are applied, and possibly also to lend a greater consistency to the end product, the material itself may include a substrate composed of a web or layer of nonwoven fabric, or even of tissue paper. A web or layer of unbonded fibers is placed on top of this substrate, on which the superabsorbent particles are distributed. Then a second web or layer of unbonded fiber is applied over the SAP particles. Alternatively, the structure may comprise a nonwoven fabric, over which a web of unbonded fiber is placed, with the superabsorbent particles distributed between these two components.

In a modified embodiment of the invention, the two fibrous webs or layers have a different titer (i.e. a fineness) of the fibers for the two layers. To be more precise, the layer or web that comes to be underneath during the manufacturing process, on which the powder of superabsorbent material is distributed, may have a prevalence of fibers with a lower titer and consequently be more compact than the layer or web that is placed on top. Typically, the fibers of the bottom layer or web, e.g. viscose and/or polyester fibers, may have a titer of 1.7 dtex or less, while the titer of the fibers for the top layer, which may again be viscose and/or polyester, for instance, may be 3.3 dtex or more. A structure of this type avoids the need to add a bottom layer of tissue paper or nonwoven fabric.

According to yet another aspect, the invention concerns an absorbent material in sheet form, comprising at least one layer of textile fibers with superabsorbent particles adhering to said layer, said particles and said layer being joined together by a bonding agent applied by impregnation, using a bonding agent that is dispersible or soluble in water, as well as a composite absorbent product including an absorbent material in sheet form of the aforesaid type among its various components.

When the rapidly water-soluble or dispersible bonding agent is mixed with a second, scarcely water-soluble or dispersible bonding agent, the latter may be, for instance, a vinyl resin of known type.

Further advantageous characteristics and embodiments of the invention are illustrated in the attached claims.

### BRIEF DESCRIPTION OF THE DRAWINGS

A better understanding of the invention can be gained from the description and the attached drawing, which shows non-restrictive practical embodiments of the invention. To be more precise, in the drawing:

FIG. 1 shows a layout of our production line that implements the present invention;

FIG. 2 is a schematic, enlarged cross-section of the material obtained using the procedure according to the invention;

FIG. 3 is a schematic cross-section of a sanitary towel made with the absorbent material manufactured according to the invention;

FIGS. 4A and 4B are two diagrams comparing the time it takes for two resins or adhesives to dissolve.

### DETAILED DESCRIPTION OF AN EMBODIMENT OF THE INVENTION

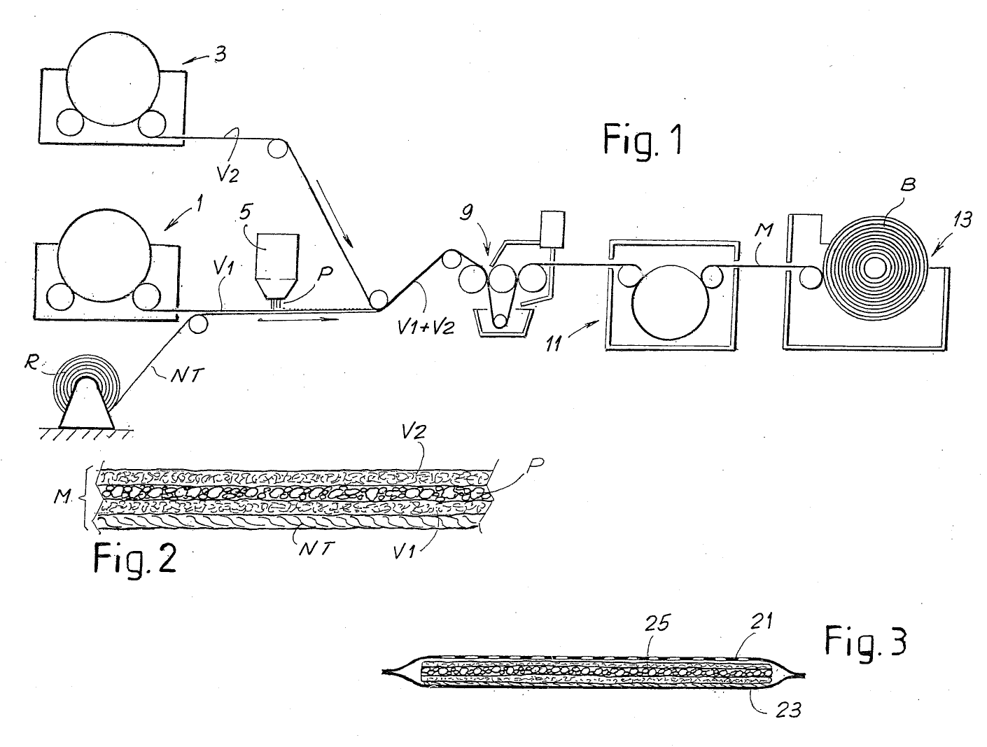

FIG. 1 shows an example of a production line for implementing the method according to the invention. In this example, there are two carding machines, indicated by numerals 1 and 3, that produce two webs or layers V1 and V2 of unbonded fibers, i.e. the fibers leaving the carding machine are not bonded together in such a way as to lend the web any significant mechanical strength. They are bonded in subsequent stages of the production process.

The web V1 is passed underneath a distributor 5, which delivers superabsorbent particles P, e.g. superabsorbent polymer particles. These particles are distributed evenly over the upper surface of the web V1. In the layout shown by way of example in FIG. 1, before the particles are distributed, the web V1 is placed on a layer of nonwoven fabrics or tissue paper NT, which is unwound by an unwinder from a roll R. The main purpose of this sheet of nonwoven fabric is to prevent the superabsorbent particles P from escaping through the relatively open structure of the fibers forming the unbonded web V1.

After the particles P have been distributed over the web V1, the second web V2, produced by the carding machine 3, is placed on top of web V1. The two webs V1+V2, one on top of the other, together with the particles P between them and the underlying sheet of nonwoven fabric NT, are delivered to an impregnation station generically indicated by numeral 9. In the example illustrated, this station is schematically in the form of a padder with three horizontal cylinders and a double impregnation tank. It may be a different machine, however, such as a two-cylinder padder, or a spraying machine, or whatever. At the station 9 the webs V1 and V2, the nonwoven sheet NT and the particles P distributed between the webs V1, V2 are impregnated with a bonding agent in the form of a foam composed of an air and water emulsion of a crosslinkable resin. The consequent swelling of the strip of material is rapidly reversed in the subsequent drying stage.

The impregnated webs V1, V2 and nonwoven sheet NT are delivered to a drying oven, schematically indicated by numeral 11. In the layout of FIG. 1, the oven includes a heated idler roll, but it is important to be aware that any other suitable type of heating or drying device may be used.

The heat delivered in the oven 11 eliminates the water from the bonding agent and crosslinks the resin. The material M emerging from the oven, comprising the webs V1 and V2, the particles P trapped between them and the sheet of nonwoven fabric NT, with the bonding agent holding these components of the material together and bonding the fibers forming the webs V1 and V2, is wound onto spools or reels B in a winding machine 13.

The structure of the finished material M is schematically represented in FIG. 2. This material can be used (alone or in combination with other layers or components) to manufacture absorbent products, such as a diaper or a sanitary towel, but it can also be used to advantage in the production of absorbent materials for the foodstuffs industry, or in domestic cleaning articles, or more in general for any product that has to have an absorbent textile structure.

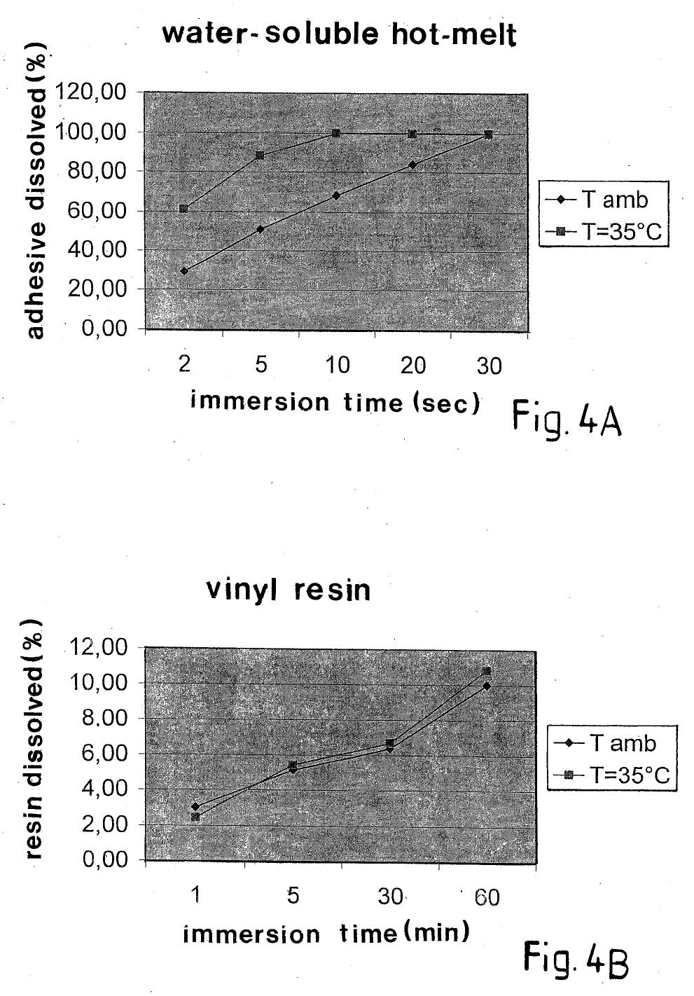

By way of example, FIG. 3 schematically illustrates the cross-section of a sanitary towel comprising an upper permeable layer (top sheet) 21, a lower impermeable layer (back sheet) 23, and an internal absorbent layer 25 composed of a length of material M, manufactured as described above.

In the example illustrated, the webs V1 and V2 are webs of unbonded carded fibers, that are bonded together by the bonding agent used to anchor the superabsorbent particles P to the webs. The webs V1 and V2 may also be of another kind, however, and they may even be different from each other. For instance, one solution could involve combining a layer of nonwoven fabric with a web of carded fiber, or combining two layers of nonwoven fabric together.

When carded webs are used, they may be made using fibers of polypropylene, polyethylene, polyester, polyamide, viscose, cotton, biological or biodegradable fibers, polylactic acid based fibers, or mixtures thereof. By way of example, the fibers may have a titer coming between 0.25 dtex and 20 dtex, and lengths ranging between 6 mm and 80 mm, for instance.

The percentages by weight of the components in the end product may vary within the above-mentioned ranges. In a particularly advantageous embodiment, the following percentages by weight can be used, expressed as percentage of the final weight of the dried end product:
superabsorbent particles 64%
bonding agent 15%
textile fibers 21%.

### Test for Determining the Water Solubility of the Bonding Agent

As mentioned earlier, one of the important aspects of the present invention consists in the solubility or dispersibility in water of the bonding agent used to anchor the superabsorbent powders to the fibers forming the textile layers or webs in the product. The method described below is used to measure the bonding agent's solubility or capacity for dispersion in water. This method is applicable to all types of material that are partially or completely soluble in water.

The following materials are required:
qty 1 500 ml beaker;
qty 1 heat-resistant adhesive tape;
qty 1 heating plate;
qty 1 thermometer;
qty 1 timer;
qty 1 oven.

At least two tests are performed for each immersion time established: each test must be conducted with a film of bonding agent 100 μm thick and 40×40 mm in size.

Before the test, the samples must be conditioned at 23° C. and 50% relative humidity for 24 hours.

The beaker is filled with 250 ml of demineralized water at a known temperature and left to rest for two minutes. A piece of adhesive tape of known weight (m_{N}) is attached to the top of a piece of water-soluble film made of the bonding agent (approximately 40×40 mm), also of exactly known weight (m_{F}), so that the tape covers approximately 1 mm said film. Then the film is immersed completely in the water for a pre-established time (t), after which the undissolved remainder of the film is withdrawn and deposited on a sheet of aluminium foil (of previously-established weight, m_{A}), which is subsequently placed in the oven and heated at 80° C. until a constant weight is reached.

This procedure is repeated for various different immersion times to enable a solubility rate curve to be charted, i.e. the percentage of bonding agent dissolving as a function of the immersion time.

### Procedure

1 Cut the film of bonding agent into a 40 mm square and record its weight, m_{F}, on a balance that guarantees a precision of 0.0005 g.
2 Cut a piece of adhesive tape 4 cm long and record its weight, m_{N}; attach it to one side of the film, making sure to cover only 1 mm max of the film's surface.
3 Cut a round sheet of aluminium foil approximately 5 cm in diameter and make a record of its weight, m_{A}.
4 Pour 250 ml of demineralized water into the beaker and, with the aid of the adhesive tape, immerse the film completely in the water, recording the immersion time (tᵢ).
5 If the test is performed at a temperature higher than 25° C., complete step 4 over the heating plate, using a thermometer to ensure that the established temperature has been reached.
6 At the end of the established immersion time, lift out the remaining film, place it, on the sheet of aluminium foil and heat it in an oven at 80° C. until it reaches a stable weight (i.e. until all the water has evaporated).
7 Repeat steps 1-5 using progressively longer immersion times, up to a maximum time Tₘₐₓ that coincides with when the film dissolves completely.

For each film, calculate the percentage of dissolved adhesive w % as follows:

$$w \% = \frac{{m}_{F} - ( {m}_{t} - {m}_{A} - {m}_{N} )}{{m}_{F}} \cdot 100$$

where:
m_{F} is the weight, expressed in grams, of the initial film of bonding agent
m_{N} is the weight, expressed in grams, of the adhesive tape
m_{A} is the weight, expressed in grams, of the aluminium foil
mₜ is the weight, expressed in grams, of the aluminium foil+the adhesive tape+the remainder of the film of bonding agent after drying in the oven,
   considering the average of the results obtained from repeating at least two tests for each immersion time.

The solubility curve is obtained by drawing a graph with the values w % (on the X axis) and the immersion times tᵢ (on the Y axis) and then interpolating the dots.

To establish which immersion times to use, immerse the film completely in water and make a note of the time Tₘₐₓ that it takes to dissolve completely (if the film is completely water-soluble; if not, establish a maximum time of 2 hours): depending on the results obtained, choose immersion times corresponding to a partial dispersion of the film that enable significant weight variations to be recorded.

FIGS. 4A and 4B compare two diagrams showing the water solubility rates of a water-soluble hot-melt adhesive (FIG. 4A) suitable for use in implementing the present invention, and a scarcely soluble vinyl resin (FIG. 4B) of the type commonly used according to the state of the art in the manufacture of absorbent products. The times in seconds are recorded on the X axis and the weight percentages of dissolved resin on the Y axis. Each diagram shows two experimental curves, determined one at ambient temperature (Tamb) and one at a temperature of 35° C.

We can see that 40% of the water-soluble hot-melt bonding agent has already dissolved after 4 seconds of being immersed in water at ambient temperature (Tamb). At a temperature of 35° C., i.e. approximately at the temperature of body fluids, it dissolves even more rapidly. On the other hand, the vinyl resins sometimes used to bond the fibers in this type of product have far slower solubility rates and can be considered substantially non-soluble in water.

Clearly the drawing shows just one practical embodiment of the invention, which may vary in shape and layout without departing from the context of the concept behind the invention.

1. Method for the manufacture of an absorbent composite material, comprising the following steps:
distributing superabsorbent particles on at least one first textile web;
impregnating the full thickness of textile web, on which said superabsorbent particles have been distributed, with a bonding agent that is highly soluble in water;
drying said bonding agent and causing adhesion of the particles to said at least one textile web.

2. Method as in claim 1, wherein said particles are placed between said first textile web and a second textile web placed over the first textile web and in wherein the full thickness of the first and second textile webs, together with the superabsorbent particles coming between them, is subsequently impregnated with said highly water-soluble bonding agent.

3. Method as in claim 1, further comprising the following steps:
providing a first web of textile fibers;
distributing a given quantity of superabsorbent particles on one side of said first web of textile fibers;
applying a second web of textile fibers over the superabsorbent particles;
impregnating the structure comprising the first textile web, the second textile web and the super absorbent particles contained between said two webs with the bonding agent;
drying or crosslinking the bonding agent.

4. Method as in claim 3, wherein said first and said second webs are unbonded fibrous webs, preferably made of carded fibers, said bonding agent bonding the fibers in said webs.

5. Method as in claim 1, wherein said textile web(s) and said superabsorbent particles are impregnated by immersion in said bonding agent, or in an emulsion of said bonding agent.

6. Method as in claim 1, further comprising applying a quantity of superabsorbent particles per unit of surface area up to 300 g/m², and preferably between 2 and 300 g/m², more preferably between 10 and 200 g/m² and even more preferably, between 10 and 100 g/m², and in particular between 50 and 100 g/m².

7. Method as in claim 1, wherein said web or each of said webs has a weight per unit of surface area between 5 and 150 g/m², and preferably between 5 and 120 g/m² and more preferably, between 8 and 100 g/m² and in particular between 8 and 80 g/m², or more specifically between 8 and 60 g/m².

8. Method as in claim 1, wherein the total weight of the web or webs, excluding the weight of the bonding agent and superabsorbent particles, is less than 150 g/m², and preferably less than 120 g/m².

9. Method as in claim 1, further comprising applying a quantity by weight of bonding agent between 5% and 50%, and preferably between 10% and 50%, and even more preferably between 15% and 35% of the weight of the end product.

10. Method as in claim 9, further comprising applying a quantity by weight of bonding agent approximately between 15% and 25% of the weight of the end product.

11. Method as in claim 1, wherein the ratio between the weight of the superabsorbent particles and the weight ofthe fibrous webs, excluding the weight ofthe bonding agent, is between 0.8:1 and 4:1, and preferably between 1:1 and 3.5:1 and more preferably between 1.5:1 and 3.5:1, and in particular between 2:1 and 3.2:1.

12. Method as in claim 1, wherein said bonding agent is composed of an emulsion of a crosslinkable resin.

13. Method as in claim 1, wherein said bonding agent is a water-based emulsion of a crosslinkable resin.

14. Method as in claim 12, wherein said bonding agent is an air and water emulsion of said crosslinkable resin.

15. Method as in one or more of the claim 10, wherein said first and possibly said second textile web(s) and the superabsorbent particles, impregnated with said crosslinkable resin, are dried in an oven to eliminate the water and complete the crosslinking of the resin.

16. Method as in claim 1, wherein said web(s) are composed of fibers whose length comes between 6 mm and 80 mm.

17. Method as in claim 16, wherein said fibers have a titer of between 0.25 dtex and 20 dtex.

18. Method as in claim 1, one first and one second of the webs being used have different fiber titers.

19. Method as in claim 18, wherein the first web, on which the superabsorbent particles are distributed, is composed of fibers with a titer mainly corresponding to approximately 1.7 dtex or less, and the second web, that is applied over the superabsorbent particles, is composed of fibers with a titer mainly corresponding to approximately 3.3 dtex or more.

20. Method as in claim 1, wherein said fibers are selected from the group comprising: polypropylene, polyethylene, polyester, polyamide, viscose, cotton, biological fibers, biodegradable fibers, polylactic acid (PLA) based fibers, or mixtures thereof.

21. Method as in claim 1, wherein said first web is placed on a supporting layer before the superabsorbent particles are distributed thereon.

22. Method as in claim 21, wherein said supporting layer is a sheet of nonwoven fabric or tissue paper.

23. Method as in claim 1, wherein said bonding agent has a water solubility such that it enables the rapid absorption of body fluids by an absorbent material manufactured with said bonding agent.

24. Method as in claim 1, wherein the bonding agent dissolves at such a rate that at least 40% of the bonding agent dissolves within a time corresponding to no more than 10 seconds, and preferably corresponding to 6 seconds or less, and even more preferably corresponding to 4 seconds or less at ambient temperature.

25. Method as in claim 1, wherein auto-expanding resin particles are added and said auto-expanding resin particles are heated to induce them to expand after the impregnation of the structure with said bonding agent.

26. Method as in claim 25, wherein said expanding resin is applied by mixing it with said bonding agent.

27. Method as in claim 1, wherein said highly water-soluble bonding agent is mixed with a bonding agent that is scarcely soluble in water.

28. Method as in claim 27, wherein the total weight of the bonding agent is composed of 50% to 100% of highly water-soluble bonding agent and 50% to 0% of scarcely water-soluble bonding agent.

29. Method as in claim 28, wherein the total weight ofthe bonding agent is composed of 70% to 90% of highly water-soluble bonding agent and 30% to 10% of scarcely water-soluble bonding agent.

30. An absorbent sheet material comprising at least one layer oftextile fibers and superabsorbent particles adhering to said layer, said particles and layer being joined together by means of a bonding agent, applied to said layer after said superabsorbent particles have been distributed thereon, that impregnates the full thickness of said at least one layer and that is highly soluble in water.

31. Absorbent material as in claim 30, further comprising a first layer of textile fibers and a second layer of textile fibers, said particles being placed between said first and said second layers of textile fibers, said first and said second layers of textile fibers and said particles being joined together by said bonding agent, that penetrates the full thickness of the first and second layers and the particles placed between them.

32. Absorbent material as in claim 30, wherein said first and possibly said second layer of textile fibers are made of a web of fibers that are bonded by means of said bonding agent.

33. Absorbent material as in claim 30, wherein said first and possibly said second layer of textile fibers are made of a nonwoven fabric.

34. Absorbent material as in claim 30, wherein said first and possibly said second layer of textile fibers are made of carded fibers.

35. Absorbent material as in claim 30, wherein the fibers of said first and/or said second layer have a length between 6 mm and 80 mm.

36. Absorbent material as in claim 30, wherein the fibers of said first and/or said second layer have a titer coming between 0.25 dtex and 20 dtex.

37. Absorbent material as in claim 30, further comprising a first layer of fibers and a second layer of fibers, between which said superabsorbent particles are placed, in which said first and said second layers contain fibers with titers that differ from one another.

38. Absorbent material as in claim 37, wherein the first layer is composed of fibers with a titer mainly corresponding to approximately 1.7 dtex or less, and the second layer is composed of fibers with a titer mainly corresponding to approximately 3.3 dtex or more.

39. Absorbent material as in claim 30, further comprising particles of an expanding resin.

40. Absorbent material as in claim 30, wherein said bonding agent dissolves at such a rate that at least 40% of the bonding agent dissolves within 10 second or less, and preferably within 6 seconds or less, and even more preferably within 4 seconds or less, at ambient temperature.

41. Absorbent material as in claim 30, further comprising a quantity of superabsorbent particles per unit of surface area of up to 300 g/m², and preferably between 2 and 300 g/m² and more preferably, between 10 and 200 g/m² and even more preferably, between 10 and 100 g/m², and in particular between 50 and 100 g/m².

42. Absorbent material as in claim 30, wherein said layer or each of said layers have a weight between 5 and 150 g/m², and preferably between 5 and 120 g/m² and more preferably between 8 and 100 g/m², and in particular between 8 and 80 g/m², or 8 and 16 g/m², excluding the weight of the bonding agent and superabsorbent particles.

43. Absorbent material as in claim 30, wherein the total weight of the fibrous layer or of each fibrous layer, excluding the weight of the bonding agent and superabsorbent particles, is less than 150 g/m², and preferably less than 120 g/m².

44. Absorbent material as in claim 30, further comprising a quantity by weight of bonding agent or adhesive between 5% and 50%, preferably between 10% and 50%, and even more preferably between 15% and 35% of the end product.

45. Absorbent material as in claim 44, further comprising a quantity by weight of bonding agent approximately between 15% and 25% of the end product.

46. Absorbent material as in claim 30, wherein the ratio between the weight of the superabsorbent particles and the weight of the fibrous webs, excluding the weight of the bonding agent, comes between 0.8:1 and 4:1, and preferably between 1:1 and 3.5:1, and more preferably between 1.5:1 and 3.5:1, and in particular between 2:1 and 3.2:1.

47. Absorbent material as in claim 30, wherein the fibers forming said first and/or said second layer are selected from the group including: polypropylene, polyethylene, polyester, polyamide, viscose, cotton, biological fibers, biodegradable fibers, polylactic acid (PLA) based fibers, or mixtures thereof.

48. Absorbent material as in claim 30, further comprising a first and a second layer of textile fibers with the superabsorbent particles placed in between them and a further layer of nonwoven fabric combined with said layers.

49. Absorbent material as in claim 30, wherein said bonding agent is polyvinyl alcohol polymer based.

50. Absorbent material as in claim 30, wherein said particles and said layer are united not only by means of a highly water-soluble bonding agent, but also by a scarcely water-soluble bonding agent.

51. Absorbent material as in claim 50, further comprising a percentage by weight of the total weight of the boding agent ranging from 50% to 100% of highly water-soluble bonding agent, and from 50% to 0% of scarcely water-soluble bonding agent, and preferably ranging from 70% to 90% of highly water-soluble bonding agent and from 30% to 10% of scarcely water-soluble bonding agent.

52. An absorbent product, comprising a permeable bottom layer (back sheet), a top layer (top sheet) permeable to fluids and an internal absorbent structure, wherein said internal absorbent structure includes at least one absorbent sheet material comprising at least one layer of textile fibers and superabsorbent particles adhering to said layer, said particles and layer being joined together by means of a bonding agent, applied to said layer after said superabsorbent particles have been distributed thereon, that impregnates the full thickness of said at least one layer and that is highly soluble in water.

53. Method as in claim 2, further comprising the following steps:
providing a first web of textile fibers;
distributing a given quantity of superabsorbent particles on one side of said first web of textile fibers;
applying a second web of textile fibers over the superabsorbent particles;
impregnating the structure comprising the first textile web, the second textile web and the super absorbent particles contained between said two webs with the bonding agent;
drying or crosslinking the bonding agent.

54. Method as in claim 53, wherein said first and said second webs are unbonded fibrous webs, preferably made of carded fibers, said bonding agent bonding the fibers in said webs.

55. Absorbent material as in claim 31, wherein said first and possibly said second layer of textile fibers are made of a web of fibers that are bonded by means of said bonding agent.

56. Absorbent material as in claim 31, wherein said first and possibly said second layer of textile fibers are made of a nonwoven fabric.

57. Absorbent material as in claim 32, wherein said first and possibly said second layer of textile fibers are made of a nonwoven fabric.

58. Absorbent material as in claim 55, wherein said first and possibly said second layer of textile fibers are made of a nonwoven fabric.
